(19) United States
(12) Patent Application Publication
(10) Pub. No.: US 2018/0361045 A1
(43) Pub. Date: Dec. 20, 2018

(54) MONITORING AND REMOTE CONTROL OF A DIALYSIS MACHINE USING CLONED OR RECONSTRUCTED INTERFACES

(71) Applicant: Fresenius Medical Care Deutschland GmbH, Bad Homburg (DE)

(72) Inventor: Erik Griessmann, Schweinfurt (DE)

(21) Appl. No.: 15/945,917

(22) Filed: Apr. 5, 2018

(30) Foreign Application Priority Data
Jun. 14, 2017 (EP) 17176139.8

(51) Int. Cl. A61M 1/16; G06F 3/0484; G06T 11/00

(52) U.S. Cl. CPC A61M 1/1601; G06F 3/0484; G06T 11/003; A61M 1/267; A61M 2205/502; A61M 2205/3584; A61M 2205/50

(57) ABSTRACT

A system and method for monitoring and/or remote control of a dialysis machine includes: providing a display device, wherein the display device comprises a display and a processor, providing graphical user interface (GUI) layout resources and/or dynamic data, interpreting the GUI layout resources and/or the dynamic data by the processor, and outputting of a screen layout by the processor. The system and method provide for monitoring and/or remote control of a dialysis machine through the use of one or more cloned or reconstructed interfaces.

### CROSS-REFERENCE TO RELATED APPLICATIONS

Priority is claimed to European Patent Application No. EP 17176139.8, filed on Jun. 14, 2017, the entire disclosure of which is hereby incorporated by reference herein.

### FIELD

Exemplary embodiments of the invention concern a method for monitoring and/or remotely controlling of a dialysis machine. Furthermore, exemplary embodiments of the invention concern a method for storing information on the state of a dialysis machine as well as a method for reconstructing or cloning (e.g., for reconstructing or cloning exactly) a screen layout or a sequence of screen layouts of a dialysis machine. Additionally, exemplary embodiments of the invention concern a display device and a dialysis machine, a digital storage medium, a computer program product and a computer program.

### BACKGROUND

Storing information on the state of a dialysis machine and reconstructing a sequence of several screen layouts of a dialysis machine frequently involves considerable amounts of data to be stored or transferred. It would be desirable to address this issue in connection with monitoring and/or remotely controlling a dialysis machine.

### SUMMARY

In an exemplary embodiment, the present invention provides a method for monitoring and/or remote control of a dialysis machine. The method includes: providing a display device, wherein the display device comprises a display and a processor; providing graphical user interface (GUI) layout resources and dynamic data; interpreting, by the processor, the GUI layout resources and the dynamic data; outputting, by the processor, a screen layout; and displaying the screen layout on the display.

In another exemplary embodiment, the present invention provides a method which includes: providing a dialysis machine with a display; providing a computer-readable medium and/or a connection to a network; and storing data on the state of the dialysis machine on the computer-readable medium and/or transferring the data into the network, wherein the data comprises screen layout data from which one or several screen layouts of the dialysis machine may be reconstructed or cloned.

In yet another exemplary embodiment, the present invention provides a method for cloning or reconstructing a screen layout or a sequence of screen layouts from a dialysis machine. The method includes: providing a visualizer; providing screen layout data from a computer-readable medium and/or from a network; processing, by the visualizer, the screen layout data; outputting a screen layout or the sequence of screen layouts; and displaying the screen layout or the sequence of screen layouts on a display of the display device or of the dialysis machine.

In yet another exemplary embodiment, the present invention provides a system. The system includes: a dialysis machine; a display device for monitoring and/or remote control of the dialysis machine, comprising a display and a processor; wherein the processor of the display device is configured to interpret graphical user interface (GUI) layout resources and dynamic data, to output a screen layout, and to cause the screen layout to be displayed on the display.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 depicts a dialysis machine and a display device;

FIG. 2 depicts a menu with two entries;

FIG. 3 depicts a dialysis machine, which is remotely controlled by a display device;

FIG. 4 depicts a reconstruction of a screen layout; and

FIG. 5 depicts, in a simplified schematic, an example for a dialysis machine according to the invention with a processor.

### DETAILED DESCRIPTION

A reduction of the quantity of data stored or transferred with respect to monitoring and/or remotely controlling of a dialysis machine, storing information on the state of a dialysis machine and reconstructing or cloning a sequence of several screen layouts of a dialysis machine may in some cases be of advantage.

Exemplary embodiments of the present invention provide a method for monitoring and/or remotely controlling, for storing information on the state of a dialysis machine and for reconstructing or cloning a sequence of screen layouts of a dialysis machine. Further exemplary embodiments of the present invention provide a dialysis machine and a display device, a suitable digital storage medium, a suitable computer program product and a suitable computer program.

The method according to an exemplary embodiment of the invention for monitoring and/or remotely controlling of a dialysis machine comprises:

providing a display device, wherein the display device comprises a display and a processor;

providing graphical user interface (GUI) layout resources and/or dynamic data;

interpreting the GUI layout resources and/or dynamic data by the processor;

outputting a screen layout by the processor.

When dialysis machines are mentioned herein, this may indicate apparatuses suitable for the treatment of blood, in particular, configured for the treatment of blood, such as hemodialysis machines, and/or other types of dialysis machines such as peritoneal dialysis machines. Furthermore, although the system described herein is discussed principally in connection with dialysis machines, it is noted that, in the scope of the present application, the system described herein may include or be used in connection with peripheral devices of a dialysis treatment and/or with other types of medical devices, such as other types of blood treatment apparatuses, including apparatuses for hemodiafiltration, filtration and/or apheresis, etc., which are suitable and/or configured for carrying out the method of the invention, as long as this does not lead to contradictions as recognized by the skilled person.

A screen layout is an arrangement of objects, which may be and/or are to be presented on a display. Such objects may comprise text, graphics and photographs, for example.

A screen layout may be shown on a suitable display. The screen layout may be presented in a format which enables to be immediately and directly displayed on a display, or it may be presented in a way which involves the translation into such a format or a suitable signal.

Screen layout data are data which enable the construction and/or reconstruction and/or cloning of screen layouts, if required or applicable, by accessing information independent of the specific treatment.

Herein, monitoring indicates passively displaying information while remotely controlling includes the ability to interact. When remotely controlling, for example, an input may be made at the display device, following which information on the input may be transmitted to the dialysis machine.

GUI layout resources are items of information which allow the processor to construct and/or reconstruction and/or clone one or several screen layouts and which are generally independent of the parameters, user interactions and measurement values of a specific treatment. The GUI layout resources may, for example, be presented as text or in binary format. In some embodiments, the GUI layout resources may be administered in a database.

Dynamic data are data which are preferably complementary to the GUI layout resources and/or are not present in the GUI layout resources. Dynamic data may, for example, be data which are only generated during the operation of the dialysis machine and/or differ generally between different patients and/or depending on different sessions, such as, for example, patient name, weight, birth date, current date and current time. This may, for example, be the case with respect to measurement values and usage/consumption values. Dynamic data may also be data which generally do not change, such as, for example, text of a usage manual.

In some embodiments, dynamic data are, for example, a number/unique identifier of a screen or a selection from a list. The identification of which list element is selected may also in some embodiments represent dynamic data. In some embodiments, alarms and/or alarm messages are dynamic data. Preferably, all possible alarm messages are known. At the occurrence of an alarm, for example, an alarm text may be associated with the alarm, for example, via an alarm number. In some embodiments, some or all of the following values represent dynamic data: the current inflow volume and/or outflow volume, the current retention time and data from device or apparatus configuration, such as sound volume, displayed language, display brightness, etc.

Dynamic data may, for example, be stored (for example in a file) and/or may be transferred via a connection or link (for example a wired or wireless connection, for example, a network connection), in particular to the display device, in particular from the dialysis machine.

In some embodiments, the transfer or provision of dynamic data occurs via a wired or wireless connection, in particular via a wired or wireless network. Dynamic data may also be stored in a configuration file, for example, when fixed screen variants are to be created, for example, in order to display information from a usage manual.

In some embodiments, the GUI layout resources comprise static GUI elements and dynamic GUI elements.

Static GUI elements are constant elements which do not change when displayed on a screen. This is preferably the case for the entire time during which the screen is displayed. Static GUI elements may, for example, be background images and/or fixed text. Fixed text may be present in different language versions.

The GUI layout resources contain, for example, formatting, text and/or graphics. Formatting comprises, for example, fonts, font sizes and/or text alignment. In addition, GUI layout resources may comprise position information which determine, for example, the position of text and/or graphics.

The GUI layout resources may comprise variants for different languages. The variants may, for example, differ in the text, formatting and/or position information. Different languages may correspond to different formatting and/or a different arrangement of the graphical elements. This may, for example, be the case for Arabic languages with a reading direction from right to left. In this case, picture alignment in a menu may, for example, be to the right instead of to the left. The formatting and/or arrangement of the graphical elements, which differs in some languages, may, for example, be read from a configuration file. This means that in some embodiments, otherwise language independent resource files may for some languages be read in additionally. In the example mentioned regarding Arabic languages, these are, for example, image data which also contain position information.

Dynamic GUI elements are elements which may take different forms or characteristics depending on a condition.

Dynamic GUI elements may, for example, be present in conjunction with a menu, in which a user may select one or several entries. Further dynamic GUI elements are present in, for example, buttons, wherein the characteristics may depend on which state a button may possess. In some embodiments, a button may possess the possible states of “pressed” or “not pressed”; alternatively, in some embodiments, the button may additionally possess the state “deactivated” or “invisible.” In addition to this, the possibility exists that a button behaves as a switch, which keeps its state after being pressed, or as a push button, which returns to the non-pressed state after release.

Dynamic GUI elements may, for example, comprise variable values. Such variable values may, for example, be states, calculated values and/or measurement values of an apparatus or device, in particular of a dialysis machine. Such variable values may, for example, be determined by a dynamic process, for example, a currently pumped volume or a measured weight. Variable values may, for example, be values entered or chosen by a user.

Dynamic GUI elements may, for example, comprise animations (for example the current image number of an image sequence) and/or progress bars.

The number of variants of screen layouts is dependent on the number of dynamic GUI elements and on the number of states which each dynamic GUI element can assume. For example, a scrollable menu with three menu elements may have six variants as each menu element may be scrolled to a given position and each respective menu element may, for example, have a button with two possible states (for example “pressed” and “not pressed”). The number of variants may also be higher, for example, when due to permission management, certain menu elements are deactivated.

The number of possible variants of screen layouts is a measure for the dynamic complexity of a screen layout and/or the presentation of the screen layout. If, for example, variable numerical values (for example a date) are shown with identical formatting, the dynamic complexity of the screen layout and/or of the presentation of the screen layout is not increased. If, however, a calendar with the names of the months is displayed, twelve variants of screen layouts and/or presentations of screen layouts exist. The dynamic complexity is important in some embodiments to be able to correctly arrange the layout of screens. Different values for a date, for example, should not lead to a superposition of GUI elements.

Translation text may in some cases be part of the GUI layout resources. In some embodiments, translation text is additionally or alternatively part of the dynamic data or stored in a further unit, for example, in a database for translation text.

The information, which is provided by the GUI layout resources and the dynamic data together, are preferably sufficient to generate and display a screen layout.

In order to display a screen layout of a dialysis machine on a remote display device, only the dynamic data have to be transmitted if the display device can access the GUI layout resources. The process of enabling and/or presenting the display of a GUI or screen layout by using or transmitting GUI layout resources and the dynamic data may, in some cases, be referred to as “reconstructing” or “cloning” and the resulting interface generated in such manner may be in some cases known as a reconstructed or cloned interface. When GUI layout resources are given, each set of dynamic data is preferably uniquely assigned to a screen layout.

According to the invention, the processor may produce one or several screen layouts from GUI layout resources and dynamic data (e.g. producing a cloned or reconstructed interface). The processor of the display device, for example, may execute software.

In some embodiments, the method for monitoring according to the invention is designed such that the processor produces screen layouts, which are identical with the screen layouts which are displayed on the dialysis machine simultaneously or almost simultaneously.

In some embodiments, the dialysis machine uses the same mechanism and/or processor for displaying screen layouts as the display device.

In some embodiments, binary GUI layout resources are used on the dialysis machine and/or the display device. The GUI layout resources are preferably convertible into Extensible Markup Language (XML) data. In some embodiments, the conversion is also possible in the opposite direction.

In some embodiments, the GUI layout resources exist in a defined form on the dialysis machine. In some embodiments, the GUI layout resources may be downloaded by the display device. Alternatively, the GUI layout resources may be administered by a server. The dialysis machine and/or the display device may, for example, be synchronized with the server regarding the GUI layout resources. Updates, in particular of the GUI layout resources, may in some embodiments be transferred via the server to the dialysis machine and/or the display device, for example.

In an exemplary embodiment, the method according to the invention for storing information comprises:

providing a dialysis machine with a processor and a display;

providing a computer-readable medium and/or a network; and

storing information regarding the state of the dialysis machine on the computer-readable medium and/or transferring the data into the network.

According to the present method, a dialysis machine stores information regarding its state on a computer-readable medium or transfers this information via a network.

Furthermore, an exemplary embodiment of the invention comprises the method of reconstructing or cloning (e.g., reconstructing or cloning exactly) a screen layout or a sequence of screen layouts of a dialysis machine, wherein the method comprises:

providing a visualizer;

providing screen layout data from a computer-readable medium and/or from a network;

processing the screen layout data by the visualizer; and

outputting a screen layout.

In some embodiments, the visualizer may process screen layout data in such a way that a screen layout results. A visualizer is in some embodiments software or dedicated hardware which produces (e.g., reconstructs or clones) a screen layout from screen layout data.

In order to verify the exact reconstruction or cloning of a screen layout, in some embodiments, a checksum or a hash value may be created by the display device, which may then be checked on the display device and/or on the dialysis machine. Preferably, a checksum or a hash value, respectively, may be created on the dialysis machine as well as on the display device, which are then compared to each other on the dialysis machine and/or on the display device. If the checksums or hash values, respectively, are identical then this is preferably seen as proof that the screen layout was reconstructed or cloned exactly, in particular pixel by pixel.

The screen layout data contain preferably only the minimally necessary data to reconstruct or clone a screen layout. In order to reduce the amount of data required for reconstructing or cloning a screen layout, the visualizer may, in some embodiments, access resources which contain treatment dependent information for producing screen layouts. The GUI layout resources are preferably present on the display device. In addition, the reconstruction or cloning of the screen layout, in some embodiments, only requires the screen number as well as the dynamic data.

In some embodiments, the visualizer is a part of the dialysis machine and/or of the display device.

In addition, exemplary embodiments of the invention comprise a dialysis machine and a display device for carrying out exemplary embodiments of the method according to the invention. Preferably, the dialysis machine and/or the display device comprise at least one screen, preferably a touchscreen or a screen with an associated key arrangement for user interaction.

A digital, in particular non-volatile, storage medium, in particular in the form of a computer-readable medium, in particular in the form of a floppy disk, compact disc (CD), digital video disc (DVD), or an erasable programmable read-only memory (EPROM), in particular with electronically or optically readable control signals, can interact with a programmable computer system such that the machine induced steps of the method according to exemplary embodiments of the invention are prompted.

The computer program product according to exemplary embodiments of the invention comprises a program code saved on a machine-readable storage medium or a signal wave for prompting the machine-induced steps of exemplary embodiments of the method according to the invention when the computer program product runs on a computer. A computer program product can be understood as, for example, a computer program which is stored on a data storage medium, an embedded system as a comprehensive system with a computer program (for example, an electronic device with a computer program), a network of computer-implemented computer programs (for example, a client-server system, a cloud computing system, etc.) or a computer on which a computer program is loaded, executed, saved or developed.

The term machine-readable storage medium, as used herein, denotes in certain exemplary embodiments according to the invention a medium, which contains data or information, which is interpretable by software and/or hardware. The medium may be a data medium such as a floppy disk, a CD, DVD, a Universal Serial Bus (USB) stick, a flashcard, a Secure Digital (SD) card and the like.

A computer program according to exemplary embodiments of the invention comprises a program code for prompting the machine-induced steps of exemplary embodiments of the method according to the invention, when the computer program runs on a computer. According to exemplary embodiments of the invention, a computer program can be understood as, for example, a physical software product ready for distribution, which contains a computer program.

All or some of the steps of exemplary embodiments of the method according to the invention, which are executed by the machine, may be prompted by exemplary embodiments of the digital storage medium according to the invention, the computer program product according to the invention and/or the computer program according to the invention. This is the case, in particular, concerning the interaction with exemplary embodiments of a display device according to the invention and a dialysis machine according to the invention.

When display devices are mentioned herein, this preferably refers to display devices which are arranged or may be arranged independently of the dialysis machine.

A display, which is firmly attached to the dialysis machine, is preferably described herein as a display of the dialysis machine.

Dialysis machine, dialysis apparatus and/or dialysis device are used synonymously herein. Also, display device, display unit and/or display apparatus are to be understood as synonyms.

In all of the implementations disclosed herein, the use of the expression “may be” or “may have” and so on, is to be understood synonymously with “preferably is” or “preferably has,” and so on, respectively, and is intended to illustrate an embodiment according to the invention.

Whenever numerical words are mentioned herein, the person skilled in the art shall recognize or understand them as indications of numerical lower limits. Unless it leads the person skilled in the art to an evident contradiction, the person skilled in the art shall comprehend the specification, for example, of “one” encompassing “at least one”. This understanding is also equally encompassed by the present disclosure as the interpretation that a numerical word, for example, “one” may alternatively mean “exactly one”, wherever this is evidently technically possible for the person skilled in the art. Both are encompassed by the present disclosure and apply herein to all used numerical words.

If a device or unit is mentioned herein, which serves to carry out a step of the method, this means that the respective device or unit is configured, programmed, provisioned and/or designed to carry out such step.

Embodiments according to the invention may possess one or several of the features mentioned above and below. Embodiments of the invention may comprise one or several of the features mentioned herein, in any combination, unless the person skilled in the art recognizes the specific combination as technically impossible.

In some embodiments screen layouts produced by the processor are displayed on a display, in particular, a display of the display device and/or a display of the dialysis machine.

The GUI layout resources may be stored, in some embodiments, on the display device and/or may be provided by a device or a network connected to the display device.

In some embodiments of the method for reconstructing or cloning a screen layout or a sequence of several screen layouts from a dialysis machine, the reconstructed or cloned screen layout may be displayed in a different language version from that of the recording dialysis machine.

In this way, for example, an application consultant may use the device in their own language and may control a dialysis machine, which is set to a language foreign to said consultant.

In some embodiments, several display devices may show the same screen layout of the dialysis machine simultaneously or almost simultaneously. In some embodiments, the screen layouts of several dialysis machines may be shown concurrently on a display of a display device.

In some cases, it may be required to translate the text of a screen layout into one or several languages; several or all screen variants of the translated language may be saved, in particular in an image file format (for example, Portable Network Graphics (PNG), JPG, Graphics Interchange Format (GIF), Tagged Image File Format (TIFF), etc.). By using a suitable (translation) software, formatting (for example, position and font size) and a superposition of text may be checked, verified and, if required, changed, without having to transfer a language variant to a dialysis machine.

A language variant may, for example, be translated online such that when a text is changed, the presentation of a screen is updated immediately. In this way, formatting mistakes and text superpositions may become visible immediately and may be corrected accordingly.

In an embodiment, a program suitable for creating and/or checking a translation (translation module) may be implemented using plug-in technology. In this embodiment, GUI layout resources and the core of the processor may be identical on the respective target device (in particular on the dialysis machine) and in the program. In some embodiments, it is possible that the screen layouts displayed by translation software are identical to the screen layouts displayed on the target device, such that assessments of the screen layouts on the target device may be reduced or omitted entirely.

In some embodiments, translations may be produced and/or checked, for example, via a web interface, a Hypertext Markup Language 5 (HTML5) smartphone app (browser app) or via a similar technology. In these cases, a translator may enter the translated text into a browser, for example. A web server may then, for example, generate the resulting screen layout, such that the translator can obtain immediate feedback. Thus, advantageously, some embodiments require no additional tool for translations and updates are available immediately to all translators. Furthermore, in some cases, it is not necessary anymore that translations of screen layouts are exchanged using files, but can instead be saved immediately on the web server and may be downloaded from there. In this way, in some cases, no tools, which may require administrator access, need to be installed and maintenance of the tools (including the distribution of updates) may be avoided.

The image files of screen layouts, in their respective language, which are generated by suitable software, may in some cases be used in instruction manuals.

In some embodiments, the present invention permits monitoring. To this effect, at least a unidirectional connection between data source, for example, the dialysis machine, and the display device, for example, a monitoring display, is utilized. Such a unidirectional connection may, for example, be carried out as a wired (for example as a direct or indirect wire connection) or wireless connection (for example as a radio link or infrared link).

Alternatively, a bidirectional connection may be used. A bidirectional connection may also be implemented as a wired (for example as a direct or indirect cable connection or via a wired network, for example, Ethernet, in particular via the internet) or wireless connection.

A wireless bidirectional connection between a data source and a display device may, for example, be implemented directly between the data source and the display device by using electromagnetic waves (e.g., radio frequency such as Bluetooth) or via infrared light. In some embodiments, a bidirectional connection between the data source and the display device may be routed via a wireless network, such as Wi-Fi, a mobile data connection (for example, General Packet Radio Service (GPRS), Enhanced Data rates for GSM Evolution (EDGE), third generation (3G) or Long-Term Evolution (LTE)) or a mesh network.

With a wireless connection, in some embodiments, parts of the data traffic may be routed through a wired network, for example, the data source and/or the display device may be connected via Wi-Fi to the internet.

In some embodiments, for example, a doctor or other caregiver, may monitor a medical treatment, in particular a dialysis treatment, via a fixed or mobile display device, for example, a smartphone, laptop or stationary computer. The display of the dialysis machine and the display of the display device for remote monitoring may preferably be synchronous or almost synchronous (e.g. substantially in real time).

In some embodiments of the monitoring, a user is exclusively or additionally informed by the display device when certain events occur (for example when a warning or an alarm on the monitored dialysis machine occurs). In this, the treatment course may, for example, be reconstructed later on the display device. For example, the graphical treatment course (for example the screen layouts passed through since treatment begin) and/or further information (for example a treatment protocol, which may be displayed during the image sequence, for example, graphically) may be shown on the display device for monitoring, when alarms occur. In the same way, in some cases, technical information may be displayed, for example, on the quantity of solution in the bags, information on blood flow or other sensor data, which may not be able to or should not be displayed on the display of the dialysis machine, for example, because of its size, when a patient is at the dialysis machine.

If a technician wants to investigate the cause of an alarm, in some embodiments, a knowledge database may additionally be queried, which provides further information on the specific alarm.

In some embodiments, such remote monitoring may advantageously be used by application consultants, who may, in this way, have access to apparatus and treatment information and may give instructions to a user on operating the device, for example, via telephone, which may then be checked by the treatment consultant.

In addition, in some embodiments, it may be advantageous to be able to monitor dialysis machines remotely during apparatus testing. In particular, this would be advantageous when tests are carried out under harsh conditions, for example, in a climate chamber.

In some embodiments, a screen layout can be retrieved at a display device for monitoring in a different language version than the language version of the original screen layout on the dialysis machine.

Preferably, language information is part of the GUI layout resources. Each alarm message preferably corresponds to a unique identification code. For the presentation of the alarm in a screen layout, preferably the alarm message, which corresponds to the identification code, is retrieved from the language text present in the GUI layout resources. As positions and formatting may also be language dependent, these are preferably also retrieved from GUI layout resources for presentation, to which end there may be a GUI resource configuration for each language.

The transferred dynamic data are preferably language independent. Preferably, each language uses the GUI layout resources of a master language (for example, for image information, screen composition description).

In some embodiments, only language dependent elements, such as text, positions and/or formatting, need to be exchanged for a different language. The GUI layout resources have to be downloaded once from the dialysis machine or another location, for example, a web server or a database etc.

To this end, the display device for monitoring may have stored translation text, for example, which it may have access to.

In some embodiments, the display device has access to translation text outside the display device, for example, via a network. Alternatively, with data transferred from the dialysis machine to the display device for monitoring, one or several language versions are transmitted, which differ from the language version set on the dialysis machine.

In certain embodiments, the transferred data contain no language version; each text merely possesses a code (which is identical for all language versions) or a hash value (for example, corresponding to one language), which is translated into a preferably configurable language version on the display device.

Alternatively, the transferred data contain one language version and a display device may access translation text, for example, in a database which is on the display device or which is accessible via a network.

Preferably, the language version is configurable on the display device. An application consultant may assist a patient by changing the language on the consultant's display device even if the patient has configured their dialysis machine to a language foreign to the consultant.

In some embodiments, the display device for remote monitoring may then serve as an automatic translator of the graphical user interface, in particular of the screen layouts.

In some cases, a browser, which is executed on a computer (for example, a stationary computer or a mobile computer, such as a tablet computer or a smartphone) may serve as a display device for monitoring. To this end, the dialysis machine may, in some embodiments, communicate with a web server or contain a web server.

In some embodiments, the display device for monitoring is suitable and/or configured to monitor several dialysis machines at the same time. In some embodiments, several dialysis machines may be monitored in a browser window or in an app.

Thus, in some embodiments, it is possible that, for example, one person may monitor all dialysis machines on a ward. Alternatively or additionally, the system tests in the production of dialysis machines may, in some embodiments, be monitored for several dialysis machines simultaneously.

In some embodiments, the dialysis machine and the display device are suitable and/or configured for the display device to remotely control the dialysis machine. For remotely controlling, a bidirectional connection is generally utilized between the dialysis machine and the display device, preferably a bidirectional connection as described herein in connection with remote monitoring.

A display device may in some embodiments comprise one or more control or input elements. In particular, control elements may be physical keys and/or switches, preferably keys, which are arranged around the display. A touchscreen may also be an input element.

In some embodiments, the method comprises transferring an input made by the user via the control element of the display device to the dialysis machine. For remote control, for example, the operation of a control element on the display device may be transferred to the dialysis machine.

If the control element is a key or a switch, then an identifier of the key or switch may be transmitted to the dialysis machine together with the state/change of state of the key or switch. The dialysis machine may then interpret the user's input and, for example, initiate programmed processes.

In some embodiments of the display device with a touchscreen, for example, when the touchscreen is touched, the coordinates of the touch may be transferred to the dialysis machine. The dialysis machine may then interpret the coordinates as the operation of a specific control element on the respective screen layout.

If the position of the key is known to the display device, the display device may then, in some embodiments, interpret the touching of the touchscreen as operating the specific control element and does not (or not only) transfer the coordinates of the touch, but (also) the interpretation of the operation of the control element. In some embodiments, several simultaneous touches and/or gestures (for example, swiping) may be transferred, for example, with a graphical user interface, which allows zooming in and out with two fingers (for example, pinch to zoom) on display content, for example, on a user manual. Such transfer of several simultaneous touches and/or gestures to dialysis machines may advantageously be possible, even if the dialysis machines do not comprise an input element, which recognizes several simultaneous touches and/or gestures, such as a multi-touch-display.

In some embodiments, the display device for monitoring and/or remote control must be explicitly authorized for remote control on the dialysis machine; alternatively, an authorization may be made exclusively on the display device.

In some embodiments, authorizations are required both on the dialysis machine as well as on the display device to enable monitoring and/or remote control.

The authorization may in some cases be carried out by the user or may exclusively by possible by the administrator. In some embodiments, an authorization may be made for all connected and/or connectable display devices.

In some embodiments, an authorization may be made for an unlimited time; in other embodiments, authorizations are only possible for a limited time period or a limited number of sessions (e.g., only for one session). In order to avoid operating errors, in some cases, for the authorization on the display device, the patient name may be transferred to the display device and/or displayed there.

In some embodiments, when remotely controlling, sound output at the apparatus may be partially or completely suppressed, such that a patient may continue to sleep, if a notification is released, which otherwise is accompanied with a sound. The sound may instead be played, in some embodiments, at the display device. In this way, for example, a patient may sleep without being woken up by the notification. Incidents may then be dealt with by the monitoring person, in some cases remotely.

In some embodiments, remote control and/or monitoring is at any one time only possible from one display device. In other cases, this is possible from two or more display devices simultaneously.

In some embodiments, several dialysis machines may be monitored and/or remotely controlled from one display device. In this way, a user may, for example, monitor several dialysis machines on one computer screen at the same time. In addition, in some cases, several dialysis machines, for example, all dialysis machines of a ward, may be remotely controlled from one computer.

In some embodiments, only one of several dialysis machines being monitored may be activated for remote control at any one time. In this way, confusion is avoided, which may otherwise occur with several dialysis machines being remotely controlled by one display device.

In other embodiments, several dialysis machines may be remotely controlled simultaneously. In some embodiments, it is possible to initiate processes in several dialysis machines (e.g., dialysis machines which are not connected to patients) with one command or operation on the display device. Such a remote control, with which several dialysis machines are operated at the same time, may preferably be used in initializations, self-tests, priming, cleaning and/or switching off. Also, in production and maintenance, remote control could be used in some embodiments. In this way, for example, several apparatuses could be tested at the same time, which would reduce personnel costs in some cases. Also, a recording of the remote control, in particular of the tests of one or several dialysis machines or of one or several dialysis treatments by dialysis machines may be performed centrally on the display device.

In some embodiments, past screen layouts may be displayed anew. Like in a video recording, the screen layouts may be stopped, played faster and fast forwarded and/or rewound. This may in some embodiments take place on the display of the dialysis machine and/or on the display device. To this end, the screen layout data may in some embodiments be stored on the dialysis machine and/or on the display device.

In some embodiments, in which screen layout data are only stored on the dialysis machine, the screen layout data may nevertheless be accessed by retrieving the respective data from the dialysis machine by the display device.

The repeated playing, faster playing, forwarding and/or rewinding, just like with a video, may in some cases be useful for a usability analysis or for an analysis of a suspected operating error. In particular, if patients cannot remember how they have operated the dialysis machine, such a replaying of screen layouts may be useful in an analysis of a suspected or actual operating error. For the purposes of training or marketing, amongst others, recorded sequences of screen layouts, which may be played like a video, may be used.

In some embodiments the sequences of screen layouts require only very little storage space. This means, for example, that the complete screen layout data of a dialysis treatment session require a storage space of only a few kilobytes. A conventional digital video of screen layouts of a treatment typically requires a storage space which is larger by several orders of magnitude. Also, storing of each screen presentation as an image file (for example, PNG, JPG, GIF, TIFF or bitmap (BMP)) would require a considerably larger storage space.

In some embodiments, the screen layout data are transferred as a text file and/or transmitted as text. The text file or the text, respectively, is preferably formatted as XML. Preferably, the screen layout data are generated on the dialysis machine in the format in which they are to be saved; in other embodiments, they are generated on the display device.

In some embodiments, the screen layout data are stored in a fixed storage space on the dialysis machine and/or on the display device. Alternatively or additionally, the screen layout data may be stored on a computer-readable medium, if such a computer-readable medium is connected to the dialysis machine and/or the display device. The computer-readable medium may be used for backups or for preservation of evidence. Alternatively or additionally, in some embodiments, the screen layout data may be printed in a raw version, for example, as text, in particular XML.

Backups of the screen layout data may in some embodiments be performed via a network connected to the dialysis machine and/or the display device. The screen layout data may in some embodiments be an event protocol.

A display device for remote control may, for example, be arranged in a fixed position, like, for example, a display device designed specifically to exclusively remotely control one or several dialysis machines, for example, with a touchscreen. In some cases, the display device for remote control may be a computer, which is installed in a fixed position, or a mobile computer (for example a laptop, a tablet computer or a smartphone), on which a special application is being executed or on which a browser is running, which may—in conjunction with a webserver—function as a display device. Such a webserver may be executed on the computer, on which the browser is running, or on the dialysis machine. In other embodiments, the webserver may be running on a third device, for example, on a dedicated server.

In some embodiments, the patient may operate the dialysis machine, to which he may, for example, be connected, via a display device for remote control, for example, via a tablet computer or a smartphone. For example, if a patient is woken up during a dialysis treatment with a dialysis machine by a notification text of the dialysis machine (for example an alarm), they can acknowledge the notification with the tablet computer or the smartphone without having to get up. Also, in some cases, the notification may be easier to read for the patient, if the display device comprises a larger display than the dialysis machine or if it allows magnifying the notification text. In this and other cases, the display device may be connected to the dialysis machine via Bluetooth or Wi-Fi.

In some embodiments, the dialysis machine may comprise a docking station for a mobile computer, for example, for a tablet computer or a smartphone. The user may then keep their computer there and, in some embodiments, charge it there. Furthermore, it is then possible, in some embodiments, to not have a display on the dialysis machine at all and to use the mobile computer as the only display device.

In some embodiments, the screen layouts and/or sequences of screen layouts, which are shown on the dialysis machine, are reproduced exactly on the display device. To this end, the display device may, for example, transfer test data to the dialysis machine, from which it may be concluded, whether the screen layouts are identical. In some embodiments, a hash value or a checksum of the screen layout may be calculated. The hash value or the checksum, respectively, may then be transferred from the display device to the dialysis machine (or vice versa) and be checked on the dialysis machine and/or the display device. In this way, it may be checked, whether the screen layouts are identical on the different devices.

In some embodiments, by checking the hash value or the checksum, respectively, it can be proven, whether two screen layouts, which are displayed on different devices, are identical, in particular, identical in each pixel. This may be relevant in security-critical applications. The hash value or the checksum, respectively, may, for example, be used to document the identity of the screen layouts within the framework of current legislation and to record them to be used as legal evidence.

In some embodiments, the invention may, amongst others, possess the following advantages: An exact reconstruction or cloning, and not just a similar reproduction, may be important for the safety and reliability of some embodiments and may simplify the verification.

The present invention is not limited to the described embodiments; these merely serve as illustration.

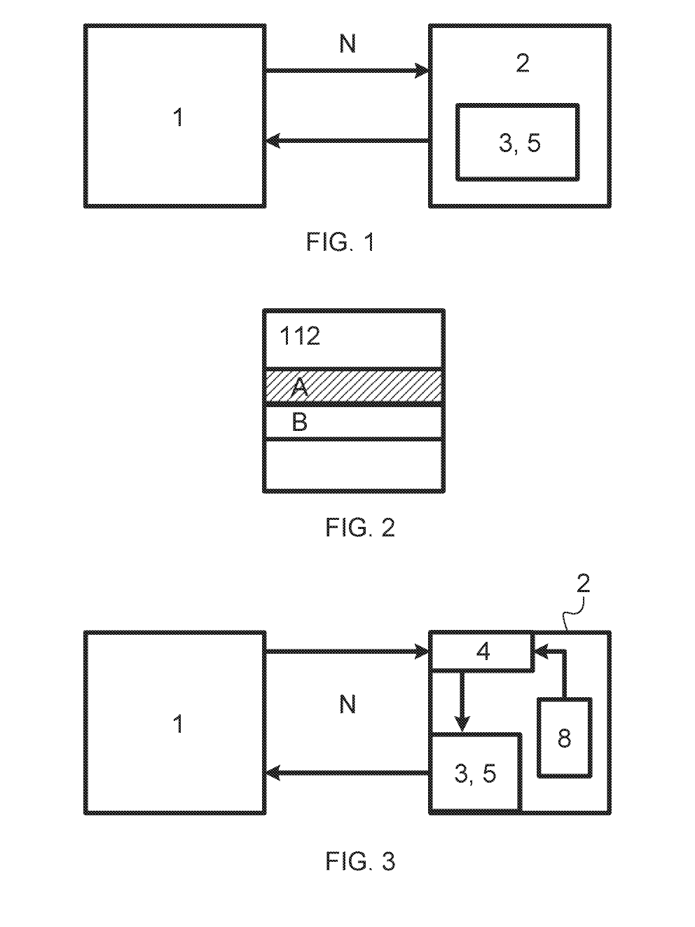

FIG. 1 shows a dialysis machine 1, which is connected to a display device 2 via a wireless network N, wherein the display device 2 comprises a display 3, which is implemented as a touchscreen 5. A patient is connected to the dialysis machine 1 for dialysis treatment. A nurse may monitor the dialysis treatment via the display device 2 and operate the dialysis machine 1 from afar via the display device 2.

FIG. 2 shows a menu with two entries (entry A and entry B). In each entry, there is one button, which may possess the state “pressed” and “not pressed”. In order to be able to reconstruct the state of the menu, for example, from stored data, one needs the screen number, the current menu position and the button state. In the example shown here, the screen (with a defined screen number, here 112) is operated, such that it is in the menu position 0 (which corresponds to the line, in which entry A is shown) and the corresponding button for entry A is pressed (the state of the button is then buttonstatus=pressed).

FIG. 3 shows a dialysis machine 1 and a display device 2 for use in exemplary embodiments of the method according to the invention for monitoring and remotely controlling. In FIG. 3, the dialysis machine 1 supplies dynamic data to the display device 2 via a network N. The dynamic data are interpreted together with GUI layout resources 8 by a processor 4 in the display device 2 and are outputted as a screen layout on the display 3 (here implemented as touchscreen 5) of the display device 2. When a user makes an input at the touchscreen 5 of the display device 2 to achieve remote control then the corresponding information, for example, the coordinates of the touch on the touchscreen 5 and/or an identification of a virtual button shown on the touchscreen 5, is transferred to the dialysis machine 1. In some cases, the transferred information causes the dialysis machine 1 to perform certain programmed actions.

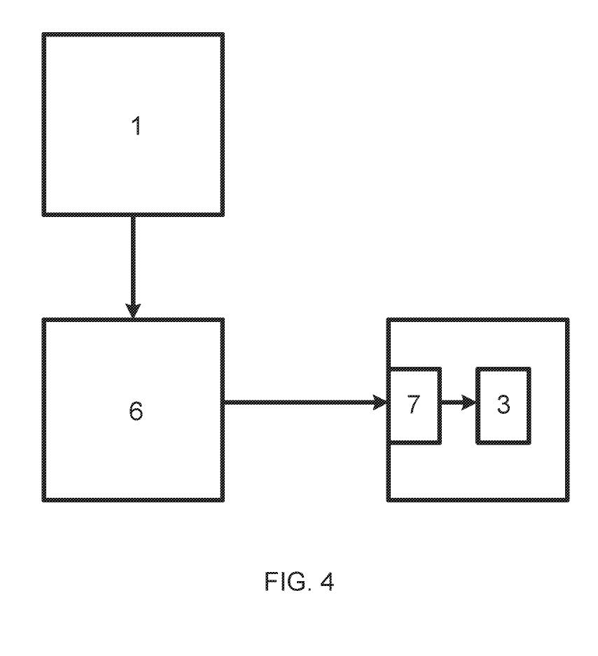

In FIG. 4, the screen layouts, which are shown on a dialysis machine 1 during a dialysis treatment, are stored as screen layout data on a computer-readable medium 6, for example, an SD card or a CD. At a later point in time, the screen layout data may be recalled from the computer-readable medium 6 by a display device 2. The screen layouts may then be reconstructed from the screen layout data with the aid of the visualizer 7 (which is implemented in software herein) and the screen layouts may be shown on the display 3 of the display device 2. The screen layouts may be shown at the actual speed of the dialysis treatment, or they may be replayed faster, stopped, fast forwarded and rewound. Alternatively, the screen layout data may be read by the dialysis machine 1 and may be reconstructed. To this end, the dialysis machine 1 also comprises a visualizer 7.

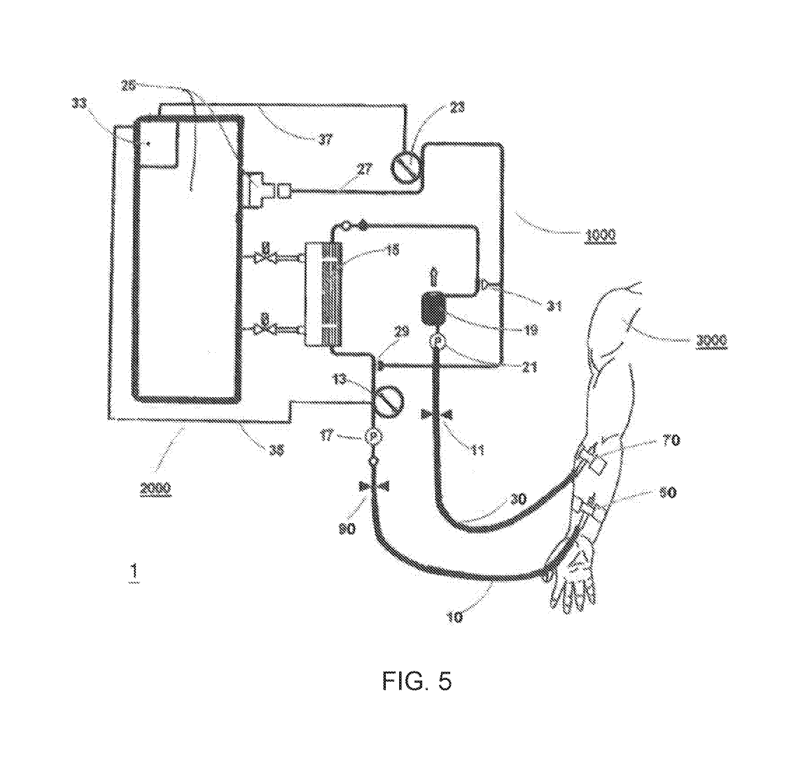

FIG. 5 shows a dialysis machine 1 according to the invention, which—as an example—is implemented as a hemodialysis machine. For this reason, among others, the components of the dialysis machines 1 mentioned in the following are to be considered optional.

In FIG. 5, an extracorporeal blood circuit 1000 is connected to a dialysis machine 1 according to the invention with a double needle access to the vascular system of a patient 3000. The extracorporeal blood circuit 1000 comprises an arterial line section 10 and a venous line section 30.

The arterial line section 10 is connected via an arterial patient access, for example, as shown in FIG. 5, in the shape of an arterial connectional needle 50, to the vascular system of the patient 3000. The venous line section 30 is connected via a venous patient access, for example, as shown in FIG. 5, in the shape of a venous connection needle 70, to the vascular system of the patient 3000.

The arterial line section 10 comprises an arterial hose clamp 90, the venous line section 30 comprises a venous hose clamp 11 or may be connected therewith, respectively.

The extracorporeal blood circuit 1000 is inserted into a blood pump 13 of the dialysis machine 1. The blood pump 13 is implemented as a roller pump, for example. The blood pump 13 conveys the blood of the patient 3000 through the extracorporeal blood circuit 1000.

The extracorporeal blood circuit 1000 comprises a blood treatment unit 15, for example, hemodialyzer, blood filter or the like.

As shown in FIG. 5, an arterial pressure sensor 17 is arranged at the arterial line section 10 downstream (referring to the typical conveying direction during a blood treatment) of the blood pump 13.

In the venous line section 30, a venous drip chamber 19 and a venous pressure sensor 21 are contained or arranged upstream of the venous drip chamber 19.

Via a second, optional conveying unit, for example, a substituate pump 23, a substituate fluid may be introduced from a substituate fluid storage unit 25 via a substituate fluid feed line 27 to a predilution addition site 29 and/or a postdilution addition site 31 for substituate fluid into the extracorporeal blood circuit 1000. The substituate pump 23 is here, by way of example, implemented as a roller pump.

The dialysis machine 1 comprises a signal processing unit 33. The signal processing unit 33 is connected to the blood pump 13 via a control line 35 and to the substituate pump 23 via a control line 37.

The signal processing unit 33 may, for example, control a pump rate or a pump power of the blood pump 13 depending on operating parameters of the substituate pump 23, for example, its pump rate or pump power.

The control lines 35 and 37 may allow bidirectional communication between the signal processing unit 33 and the blood pump 13 or the substituate pump 23, respectively.

FIG. 5 shows a connection configuration of the dialysis machine 1 according to the invention with the vascular system of the patient 3000. It will be appreciated that such a connection configuration is merely a specific exemplary embodiment.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A method for monitoring and/or remote control of a dialysis machine, comprising:
providing a display device, wherein the display device comprises a display and a processor;
providing graphical user interface (GUI) layout resources and dynamic data;
interpreting, by the processor, the GUI layout resources and the dynamic data;
outputting, by the processor, a screen layout; and
displaying the screen layout on the display.

2. The method according to claim 1, wherein the GUI layout resources are stored on the display device and/or are supplied via a network connected to the display device.

3. The method according to claim 1, wherein the dynamic data are supplied from the dialysis machine via a wired connection or a wireless connection.

4. The method according to claim 3, wherein the dynamic data are supplied via a network connection of the dialysis machine.

5. The method according to claim 1, wherein the screen layout displayed on the display is a cloned interface that is substantially the same as a screen layout on the dialysis machine.

6. The method according to claim 5, wherein the screen layout on the dialysis machine and the screen layout of the display are cloned substantially in real time, wherein a change in the screen layout on the dialysis machine is shown substantially in real time on the display.

7. The method according to claim 1, wherein the display device comprises one or more input elements; and
wherein the method further comprises: transferring an input made by a user at the one or more input elements of the display device to the dialysis machine.

8. The method according to claim 1, wherein the display device is authorized on the dialysis machine for the monitoring and/or remote control.

9. A method, comprising:
providing a dialysis machine with a display;
providing a computer-readable medium and/or a connection to a network; and
storing data on the state of the dialysis machine on the computer-readable medium and/or transferring the data into the network, wherein the data comprises screen layout data from which one or several screen layouts of the dialysis machine may be reconstructed or cloned.

10. The method according to claim 9, wherein the data is stored as a text file and/or is transferred as text.

11. A method for cloning or reconstructing a screen layout or a sequence of screen layouts from a dialysis machine, comprising:
providing a visualizer;
providing screen layout data from a computer-readable medium and/or from a network;
processing, by the visualizer, the screen layout data;
outputting a screen layout or the sequence of screen layouts; and
displaying the screen layout or the sequence of screen layouts on a display of the display device or of the dialysis machine.

12. The method according to claim 11, wherein displaying the sequence of screen layouts allows for a fast playback of the screen layouts and/or fast forwarding and/or rewinding.

13. A system, comprising:
a dialysis machine;
a display device for monitoring and/or remote control of the dialysis machine, comprising a display and a processor;
wherein the processor of the display device is configured to interpret graphical user interface (GUI) layout resources and dynamic data, to output a screen layout, and to cause the screen layout to be displayed on the display.

14. The system according to claim 13, wherein the display device is configured to store the GUI layout resources; or
wherein the display device is configured to receive the GUI layout resources via a network.

15. The system according to claim 13, wherein the display device is configured to receive the dynamic data from the dialysis machine via a wired connection or a wireless connection.

16. The system according to claim 15, wherein the dialysis machine is configured to supply the dynamic data via a network connection of the dialysis machine.

17. The system according to claim 13, wherein the screen layout displayed on the display is a cloned interface that is substantially the same as a screen layout on the dialysis machine.

18. The system according to claim 17, wherein the display device is configured to clone the screen layout of the dialysis machine substantially in real time such that a change in the screen layout on the dialysis machine is shown substantially in real time on the display of the display device.

19. The system according to claim 13, wherein the display device comprises one or more input elements; and
wherein the display device is further configured to transfer an input made by a user at the one or more input elements of the display device to the dialysis machine.

20. The system according to claim 13, wherein the dialysis machine is configured to facilitate authorization of the display device for the monitoring and/or remote control.
